(19) United States
(12) Patent Application Publication
(10) Pub. No.: US 2022/0113268 A1
(43) Pub. Date: Apr. 14, 2022

(54) LOW-COST QUANTITATIVE PHOTOTHERMAL GENETIC DETECTION OF PATHOGENS ON A PAPER HYBRID DEVICE

(71) Applicant: Board of Regents, The University of Texas System, Austin, TX (US)

(72) Inventors: XiuJun Li, El Paso, TX (US); Wan Zhou, El Paso, TX (US)

(21) Appl. No.: 17/450,049

(22) Filed: Oct. 5, 2021

Related U.S. Application Data
(60) Provisional application No. 63/087,709, filed on Oct. 5, 2020.

(51) Int. Cl. G01N 25/22; B01L 3/00; C12Q 1/6816

(52) U.S. Cl. CPC G01N 25/22; B82Y 5/00; C12Q 1/6816; B01L 3/502715

(57) ABSTRACT

A low-cost photothermal biosensing method and apparatus for the quantitative genetic detection of pathogens such as MTB DNA on a paper hybrid device using a thermometer. First, DNA capture probes were simply immobilized on paper through a one-step surface modification process. After DNA sandwich hybridization, oligonucleotide-functionalized gold nanoparticles (AuNPs) were introduced on paper and then catalyzed the oxidation reaction of 3,3′,5,5′-tetramethylbenzidine (TMB). The produced oxidized TMB, acting as a strong photothermal agent, was used for the photothermal biosensing of MTB DNA under 808 nm laser irradiation. Under optimal conditions, the on-chip quantitative detection of the target DNA was readily achieved using an inexpensive thermometer as a signal recorder. Illustrative embodiments do not require any expensive analytical instrumentation, but can achieve higher sensitivity and there are no color interference issues, compared to conventional colorimetric methods.

### CROSS REFERENCE TO RELATED APPLICATION

Referring to the application data sheet filed herewith, this application claims a benefit of priority under 35 U.S.C. 119(e) from co-pending provisional patent application U.S. Ser. No. 63/087,709, filed Oct. 5, 2020, the entire contents of which are hereby expressly incorporated herein by reference for all purposes.

### BACKGROUND OF THE INVENTION

### 1. Field of the Invention

The invention generally relates to the detection of pathogens. More particularly, illustrative embodiments are directed to a method and apparatus for the quantitative genetic detection of pathogens such as MTB DNA on a paper hybrid device using a thermometer.

### 2. Description of the Related Art

Many pathogens frequently cause global health concerns. Tuberculosis (TB), one of the deadliest infectious diseases, remains a leading cause of death from a single infection across the world. High morbidity and mortality of TB pose a significant threat to public health, causing nearly 1.5 million deaths annually.

TB is caused by a species of pathogenic bacteria, Mycobacterium tuberculosis (MTB), which has been traditionally diagnosed via time-consuming clinical examination, sputum smear microscopy, and culture of MTB bacteria. Recent years have seen a rapid development of laboratory diagnostics for TB based on molecular tests, typically MTB DNA detection methods, which significantly facilitate early diagnosis of TB, especially for latent infection. Latent TB usually happens at an early stage of infection, where MTB is internalized into the phagosomes of host macrophages and exhibits latency. However, the latent TB becomes active when MTB starts to replicate after rupturing the phagosomal membranes and translocating into the cytosol. Researchers have found two types of genes (EsxA and EsxB) and their encoding secreted proteins (6-kDa early secreted antigenic target or ESAT-6, and 10-kDa culture filtrate protein or CFP-10) play an important role in the transition from latent TB to active. Therefore, these genes can be used as specific targets for MTB DNA detection.

To date, various MTB DNA detection methods have been developed, including colorimetry, electrochemistry, fluorescence, chemiluminescence, etc, which generally rely on DNA amplification techniques, such as polymerase chain reaction (PCR), and loop-mediated isothermal amplification (LAMP). For instance, clinical samples were detected quantitatively based on the colorimetric method using PCR-amplified MTB DNA, which was based on the target-induced nanoprobe aggregation.

### SUMMARY

Illustrative embodiments provide a low-cost photothermal biosensing method for the quantitative genetic detection of pathogens such as MTB DNA on a paper hybrid device using a thermometer. First, DNA capture probes were simply immobilized on paper through a one-step surface modification process. After DNA sandwich hybridization, oligonucleotide-functionalized gold nanoparticles (AuNPs) were introduced on paper and then catalyzed the oxidation reaction of 3,3′,5,5′-tetramethylbenzidine (TMB). The produced oxidized TMB, acting as a strong photothermal agent, was used for the photothermal biosensing of MTB DNA under 808 nm laser irradiation. Under optimal conditions, the on-chip quantitative detection of the target DNA was readily achieved using an inexpensive thermometer as a signal recorder. Illustrative embodiments do not require any expensive analytical instrumentation, but can achieve higher sensitivity and there are no color interference issues, compared to conventional colorimetric methods. The method was further validated by detecting genomic DNA with high specificity. Illustrative embodiments provide photothermal biosensing for quantitative nucleic acid analysis on microfluidics using a thermometer, which brings new inspirations on the development of simple, low-cost, and miniaturized photothermal diagnostic platforms for quantitative detection of a variety of diseases at the point of care.

According to an embodiment of this disclosure, a method of quantitative genetic detection of a pathogen, comprises: immobilizing a genetic capture probe on a substrate; capturing genetic material from the pathogen using the genetic capture probe; performing sandwich hybridization of the genetic capture probe, the captured genetic material and a detector probe further comprising a nanomaterial catalyst to form a conjugate; contacting the conjugate with a photothermal agent; oxidizing the photothermal agent using the nanomaterial catalyst conjugated on the detector probe to form an oxidized photothermal agent; exposing the oxidized photothermal agent to actinic energy; and measuring a temperature increase caused by heat from the exposed oxidized photothermal agent using a thermometer to quantify the pathogen.

According to another embodiment of this disclosure, an apparatus for quantitative genetic detection of a pathogen, comprises: a substrate; a genetic capture probe immobilized on the substrate; genetic material from the pathogen captured by the genetic capture probe; a detector probe sandwich hybridized with the captured genetic material from the pathogen and the capture probe, the detector probe further comprising a nanomaterial catalyst to form a conjugate; a photothermal agent oxidized using the nanomaterial catalyst conjugated on the detector probe; a laser configured to expose the oxidized photothermal agent to actinic energy; and a thermometer configured to measure a temperature increased cause by heat from the exposed oxidized photothermal agent to quantify the pathogen.

According to another embodiment of this disclosure, a device for quantitative genetic detection of a pathogen, comprises: a substrate; a genetic capture probe immobilized by the substrate; genetic material from the pathogen captured on the genetic capture probe; a detector probe sandwich hybridized with the captured genetic material from the pathogen and the capture probe, the detector probe further comprising a nanomaterial catalyst to form a conjugate; and a photothermal agent oxidized using the nanomaterial catalyst conjugated on the detector probe.

The descriptions of the various embodiments of the present invention have been presented for purposes of illustration, but are not intended to be exhaustive or limited to the embodiments disclosed. Not all embodiments will include all of the features described in the illustrative examples. Further, different illustrative embodiments may provide different features as compared to other illustrative embodiments. Many modifications and variations will be apparent to those of ordinary skill in the art without departing from the scope and spirit of the described embodiment. The terminology used herein was chosen to best explain the principles of the embodiment, the practical application or technical improvement over technologies found in the marketplace, or to enable others of ordinary skill in the art to understand the embodiments disclosed here.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 is a schematic illustration of the working principle of AuNP-mediated photothermal biosensing of MTB target DNA on a paper hybrid device using a thermometer in accordance with an illustrative embodiment;

FIGS. 2A-2B are illustrations of results of feasibility tests of the AuNP-mediated photothermal biosensing method, (FIG. 2A) UV-vis spectra and (FIG. 2B) temperature increases of different components in the AuNP-catalyzed TMB oxidization reaction system, including the citrate buffer as blank, (a) TMB, (b) H₂O₂, (c) AuNPs, (d) TMB+H₂O₂, (e) AuNPs+H₂O₂, (f) TMB+AuNPs, and (g) AuNPs+TMB+H₂O₂, insets are photographs of the above samples, the laser power density was 0.16 W/mm², and the irradiation time was 5 minutes, error bars indicate standard deviations (n=6);

FIGS. 3A-3C are illustrations of TMB concentration optimization in the AuNP-catalyzed TMB oxidization reaction system, (FIG. 3A) UV-vis spectra, (FIG. 3B) absorbances at 650 nm and 810 nm, and (FIG. 3C) on-chip temperature measurement of reaction solutions with different TMB concentrations, the laser power density was 0.16 W/mm², and the irradiation time was 5 minutes, error bars indicate standard deviations (n=3);

FIGS. 4A-4C are illustrations of AuNPs concentration optimization in the AuNP-catalyzed TMB oxidization reaction system, (FIG. 4A) UV-vis spectra, (FIG. 4B) absorbances at 650 nm and 810 nm, and (FIG. 4C) on-chip temperature measurement of reaction solutions with different AuNPs concentrations, the laser power density was 0.16 W/mm², and the irradiation time was 5 minutes, error bars indicate standard deviations (n=3);

FIGS. 5A-5B are illustrations of kinetic studies in the photothermal biosensing process, (FIG. 5A) dynamic temperature measurement of the control (containing 0 μM target DNA) and the sample (containing 10 μM target DNA) under continuous laser irradiation, the laser power density was 0.16 W/mm², (FIG. 5B) schematic illustration of competitive effects between heat generation and heat loss during the photothermal biosensing process;

FIG. 6 is an illustration of quantitative photothermal biosensing of MTB DNA on the paper hybrid microfluidic device using a thermometer, the calibration curve of temperature increase is plotted versus the logarithmic concentration of target MTB ssDNA in the range of 0.1 to 50 μM, insets are photographs of biosensing samples at the target concentrations of (a) 0 μM, (b) 50 μM, and (c) 50 μM using blood-mimicking dye solutions (scale bar: 5 mm), the laser power density was 0.16 W/mm², and the irradiation time was 3 minutes, the error bars indicate standard deviations (n=6);

FIG. 7 is an illustration of specificity tests of the photothermal biosensing of genomic MTB DNA on the paper hybrid microfluidic device using a thermometer, temperature increases of samples containing water as blank, PBS buffer, TB knockout DNA (50 μg/mL), M. smegmatis DNA (50 μg/mL), mixture species (M. smegmatis and TB knockout, at a final DNA concentration of 50 μg/mL), M. marinum DNA (50 μg/mL), and genomic MTB DNA (25 μg/mL), the laser power density was 0.16 W/mm², and the irradiation time was 3 minutes, error bars indicate standard deviations (n=6), statistical significance was calculated using Student's t-test; ns indicates not significant between the two groups, p>0.05;

FIG. 8 is a schematic illustration of the AuNP-catalyzed TMB oxidization reaction; and

FIG. 9 is an illustration of UV-vis spectra of the bare AuNPs and the as-produced DNA probe-AuNP conjugates.

FIG. 10 is an illustration of a flow diagram of a process that can be implemented by a computer program.

### DETAILED DESCRIPTION

The illustrative embodiments recognize and take into account one or more different considerations. For example, the illustrative embodiments recognize and take into account that current MTB DNA detection methods require expensive analytical instruments and professional operators, which have significantly increased the complexity and cost of TB diagnoses and limited their wide accessibility, especially in low-resource settings. Therefore, it is still challenging and demanding to develop new detection strategies for low-cost and quantitative detection of MTB DNA.

Recently, nanomaterial-mediated photothermal biosensing methods have emerged as an attractive strategy in the quantitative detection of biomolecules, due to the simplicity in the experimental process (such as no need for pneumatic pumps), low cost in data recoding (only using a thermometer as a signal reader), and great convenience in analyzing biosensing signals (temperature-based readouts). Several photothermal biosensing platforms have been developed for the detection of biomolecules. By converting the traditional immunosensing signals to photothermal signals (i.e., temperature), biomolecules are quantified by only using a common thermometer. For instance, a photothermal immunoassay using a common thermometer for quantitative cancer biomarker detection has been developed. However, most of the current photothermal biosensing strategies have focused on protein analysis, while photothermal genetic analysis is rarely reported.

Microfluidic lab-on-a-chip (LOC) technology has provided a promising point-of-care (POC) diagnostic tool for various diseases, owing to its miniaturization, portability, low reagent consumption, etc. Among numerous LOC devices, paper-based microfluidic devices have attracted much attention given the merits of paper substrates, such as the extremely low cost, ease of manipulation, and 3D porous microstructures with a high surface-to-volume ratio. Particularly, by integrating them with other materials, such as rigid polymers, the obtained paper/polymer hybrid microfluidic devices have been capable of meeting assorted requirements for sample immobilization, fluid processing, and signal analyzing, which are suitable for easy and inexpensive nucleic acid analysis at the point of care. For example, a simple, low-cost, and versatile paper-based device has been developed for genetic analysis via a one-step surface modification method using 3-aminopropyl trimethoxysilane (APTMS). The nonfunctionalized DNA probes are directly immobilized on paper through ionic interaction between the negatively charged DNA probes and positively charged paper surface. Enhanced DNA immobilization efficiency and detection sensitivity are obtained using the paper substrate. However, this low-cost paper-based microfluidic platform has not been integrated with photothermal biosensing for quantitative DNA detection. Illustrative embodiments provide a new photothermal biosensing method on a paper hybrid microfluidic device for the low-cost quantitative detection of MTB DNA using a thermometer. Target MTB DNA (derived from the MTB EsxA gene) is recognized via the sandwich hybridization between capture DNA probes and AuNP-modified detector probes, where the former is immobilized on the paper substrate after one-step surface modification. The paper substrate can be located within a paper hybrid microfluidic device. The near-infrared (NIR) photothermal agent, oxidized 3,3′,5,5′-tetramethylbenzidine (ox-TMB), is then produced based on the AuNP-catalyzed TMB oxidization reaction, which further converts target concentration information to temperature readouts under the irradiation of an 808 nm laser. In general, embodiments of this disclosure can use one or more lasers that generate near infrared laser irradiation. By only using a thermometer, the quantification of target DNA is achieved from the on-chip temperature measurement.

Illustrative embodiments integrate the photothermal biosensing strategy on a paper hybrid microfluidic device for simple, low-cost, and quantitative detection of DNA. In comparison with conventional colorimetric methods, illustrative embodiments provide higher sensitivity with no issues of color interference, while preventing the need for advanced analytical instruments.

Following, without limitation, are examples of materials and instruments used in illustrative embodiments. Illustrative embodiments are not limited to the particular materials and instruments and sources thereof listed herein.

Whatman No. 1 chromatography paper, gold nanoparticles (with the diameter of 20 nm), 3,3′,5,5′-tetramethylbenzidine (TMB), tris (2-carboxyethyl) phosphine hydrochloride (TCEP), (3-aminopropyl) trimethoxysilane (APTMS), bovine serum albumin (BSA), saline-sodium citrate (SSC) buffer (20×, pH 7.0), sodium dodecyl sulfate (SDS), and phosphate-buffered saline (PBS, 10 mM, pH 7.4) purchased from Sigma (St. Louis, Mo., USA). Hydrogen peroxide (H₂O₂, 30% w/w) purchased from Fisher Scientific (Hampton, N.H., USA). Poly (methyl methacrylate) (PMMA, 2.0 mm in thickness) sheets purchased from Mcmaster-Carr (Los Angeles, Calif., USA). All chemicals used as received without further purification. All buffer solutions prepared by diluting in PBS buffer, including the washing buffer (2×SSC, 0.1% SDS) and the hybridization buffer (5×SSC, 0.1% SDS, 1% BSA).

Synthetic oligonucleotide sequences were purchased from Integrated DNA Technologies (Coralville, Iowa, US) and listed in Table Sl. The genomic nucleic acid species were provided by Prof. Jianjun Sun's lab (UTEP), including Mycobacterium tuberculosis (MTB), Mycobacterium smegmatis (M. smegmatis), Mycobacterium marinum (M. marinum), and MTB (ΔEsxAB) (the MTB strain with deletion of EsxB:EsxA operon, denoted as TB Knockout herein). The concentrations of DNA samples were determined via a NanoDrop spectrophotometer (Sigma-Aldrich, St. Louis, Mo., USA).

UV-vis characterization was performed on a microplate reader (Molecular Devices, LLC, Sunnyvale, Calif., US). The 808 nm diode laser (Model MDL-808, Opto Engine, Midvale, Utah, US) was used to irradiate samples. The on-chip temperature measurement was obtained by using a digital thermometer (e.g. Model 421502, Extech Instruments Corporation, US). The thermometer has a resolution of 0.1° C. and was used as a signal recorder for the following photothermal biosensing process.

The DNA probe-AuNP conjugates were prepared freshly modified from a published procedure via the typical salt-aging method. Firstly, 3 μL of 100 μM thiolated DNA (SH-DNA) probes were added into a TCEP aqueous solution (6 μL, 100 μM), followed by incubation at room temperature for 30 min. The mixture was then added to 1.0 mL of AuNPs (1.2 nM) and incubated overnight. Aliquots of 120 μL 1% SDS and 12 μL 2 M NaCl were added to the suspension slowly, followed by further incubation for 24 h. The obtained suspension was centrifuged at 13000 rpm for 20 min and washed three times with the washing buffer. The pellet was finally dispersed in PBS buffer (10 mM, pH 7.4, 150 mM NaCl, 0.1% SDS) and stored at 4° C. The synthesized DNA probe-AuNP conjugates were characterized via UV-vis spectroscopy, and the concentration of AuNPs was determined using the Beer-Lambert law.

The paper/polymer hybrid microfluidic device was designed with the Adobe AI software and fabricated using chromatography paper and PMMA sheets. Essentially, PMMA sheets were laser ablated using a laser cutter (Epilog laser, Golden, Colo.), yielding six reservoirs with a diameter of 3.5 mm and a depth of 1.5 mm for each. The chromatography paper was cut on the laser cutter to form circular regions with a diameter of 3.5 mm, and then inserted into PMMA reservoirs. The whole size of the paper/PMMA hybrid device was 75 mm×18 mm.

To immobilize capture probes on the paper substrate, a surface modification process was adapted based on a reported method. Firstly, 10 μL of 5% APTMS was added to each paper reservoir and incubated for 10 min. After washing thoroughly, the device was dried under ambient temperature. On each APTMS-modified detection zone, 5 μL of 1 μM capture probes were added and incubated for 30 min at 37° C. A BSA solution (3%, w/v) was then added as the blocking reagent and incubated for 10 min at 37° C. The DNA probe-AuNP conjugates and target MTB DNA with varying concentrations were mixed in a volume ratio of 1:1 and prehybridized for 30 min at 37° C. The obtained solution was added to the device with 10 μL per reservoir and incubated for 30 min at 37° C. Notably, washing steps were performed after each incubation step to remove nonspecific binding. Additionally, when using genomic DNA, the samples were firstly denatured at 95° C. for 5 min and then placed on ice for 1 min before the prehybridization step.

After DNA hybridization, the substrate mixture containing TMB (0.25 mg/mL), H₂O₂ (1.25 M), and the citrate buffer, was added (20 μL per reservoir) and allowed to react for 20 min at room temperature. The 808 nm laser was then used to irradiate each reservoir with a power density of 0.16 W/mm². The irradiation setup was carefully adjusted to achieve comparable sizes between the NIR laser spot and detection reservoirs with a diameter of 3.5 mm. On-chip temperature measurement was conducted using the thermometer immediately after irradiation. The position of the digital thermometer with a miniaturized probe tip (1.0 mm of diameter) was fixed in all photothermal biosensing process to avoid temperature variations due to position changes.

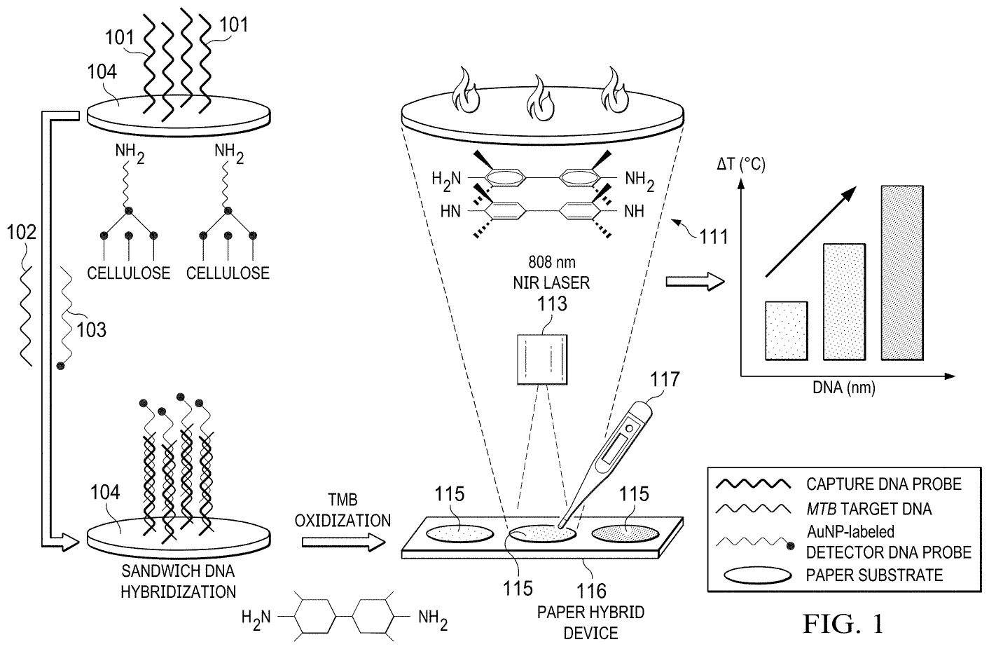

The working principle for photothermal detection of MTB DNA on a paper hybrid device 116 is shown in FIG. 1. Essentially, the capture probes 101 are first immobilized on APTMS-modified paper reservoirs (with amine groups) via ionic interaction between the positively charged paper surface and negatively charged DNA probes. When adding target sequences, DNA sandwich hybridization occurs among capture probes 101, target DNA 102, and AuNPs-labeled detector probes 103. As such, the AuNPs are immobilized on paper 104. Upon the addition of the substrate and TMB, AuNPs catalyze the oxidization reaction of TMB in the presence of H₂O₂ due to the peroxidase-like activity.

As illustrated in FIG. 1, the ox-TMB 111 is then produced with an obvious color change from colorless to blue via the one-electron charge transfer process, which can be visualized by the naked eye. Importantly, the ox-TMB 111 is a strong NIR photothermal probe, which is able to efficiently convert photon energy to thermal energy. Under the irradiation of an 808 nm laser 113, the temperature of reservoirs 115 increases and can be recorded using a thermometer 117. When increasing concentrations of the target DNA 102, more AuNPs are captured on paper 104 via DNA hybridization, thereby producing more ox-TMB with darker colors, resulting in higher temperature increase. Therefore, the temperature signals can be correlated with the target concentrations, and the photothermal biosensing can be achieved for the visual quantitative detection of MTB DNA on the paper hybrid device using the thermometer 117.

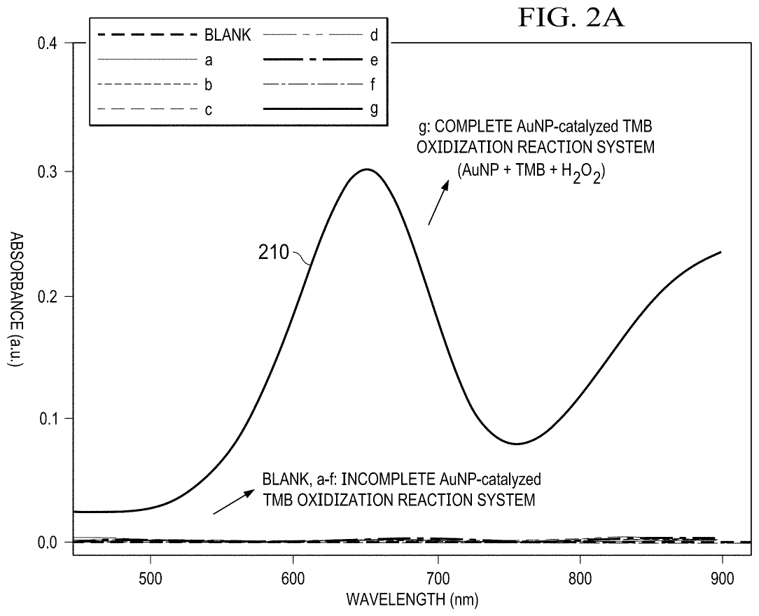

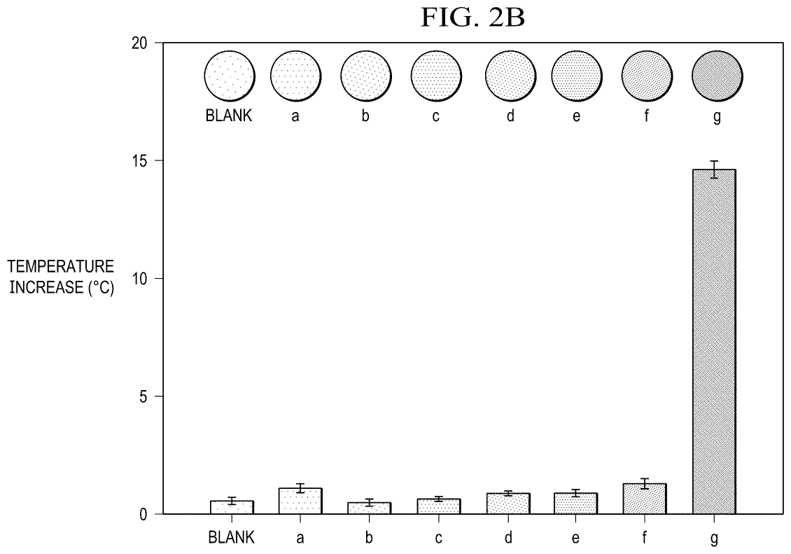

The feasibility of the AuNP-mediated photothermal biosensing method in accordance with an illustrative embodiment was investigated by testing different components in the system, and the results are shown in FIGS. 2A-2B. Referring to FIG. 2A, samples (a-g) contained different components in the AuNP-catalyzed TMB oxidization reaction system, including the citrate buffer as blank, (a) TMB, (b) H₂O₂, (c) AuNPs, (d) TMB+H₂O₂, (e) AuNPs+H₂O₂, (f) TMB+AuNPs, and (g) AuNPs+TMB+H₂O₂. All components were added at the same concentrations in all samples, namely, 0.25 mg/mL for TMB as a chromogenic substrate, 1.25 M for H₂O₂ as an oxidizing agent, and 0.03 nM for AuNPs as the catalyst. In general, embodiments of this disclosure can utilize a nanomaterial catalyst that includes at least one member selected from the group consisting of oligonucleotide-functionalized gold nanoparticles (AuNPs), oligonucleotide-functionalized iron nanoparticles (Fe₃O₄NPs), oligonucleotide-functionalized platinum nanoparticles (PtNPs).

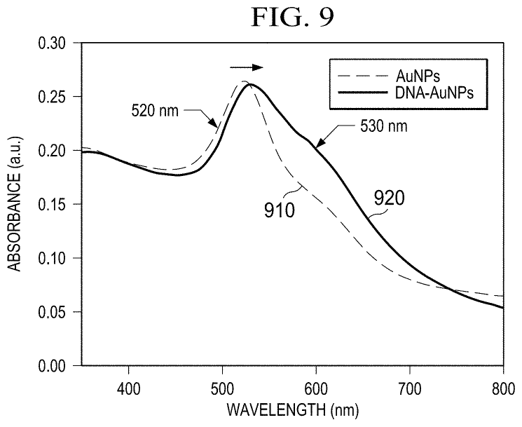

No obvious differences were observed in the UV-vis spectra and photographs of the Samples (a-f) (containing incomplete component combinations in the AuNP-catalyzed TMB oxidization reaction system), whereas an absorption peak 210 at around 650 nm appeared in Sample (g) (containing all components in the AuNP-catalyzed TMB oxidization reaction system) with a clear blue color. It is noted that the characteristic peak of AuNPs (20 nm) at 520 nm is not shown in Sample (c) due to the extremely low concentration (i.e., 0.03 nM), as compared to AuNPs at a higher concentration (i.e., 0.8 nM) in FIG. 9, showing a typical peak at 520 nm. Referring again to FIGS. 2A-2B, the result was consistent with previous studies and confirmed the formation of the oxidized product, ox-TMB, with the characteristic absorption peak at around 650 nm. Furthermore, comparing results from Sample (d) with (g), it was found that the AuNPs were capable of facilitating the oxidization reaction of TMB in the presence of H₂O₂, confirming the peroxidase-mimicking property of AuNPs. Referring to FIG. 2B, under the irradiation of the 808 nm laser, a significant temperature elevation of nearly 15.0° C. was observed in Sample (g), indicating the strong photothermal conversion efficiency of the ox-TMB, which was attributed to the strong absorption in the NIR region. Contrarily, negligible temperature increases were recorded in other samples. The results showed that temperature changes were only derived from the ox-TMB production, and there was little interference from other components in the on-chip photothermal measurements, confirming the feasibility of the AuNP-mediated photothermal detection method.

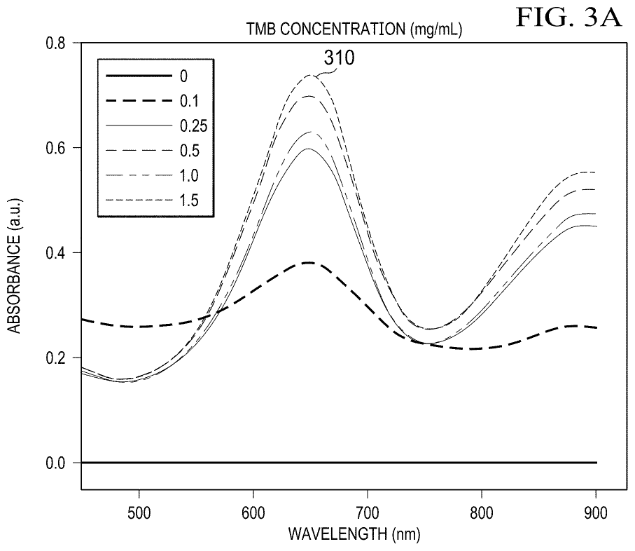

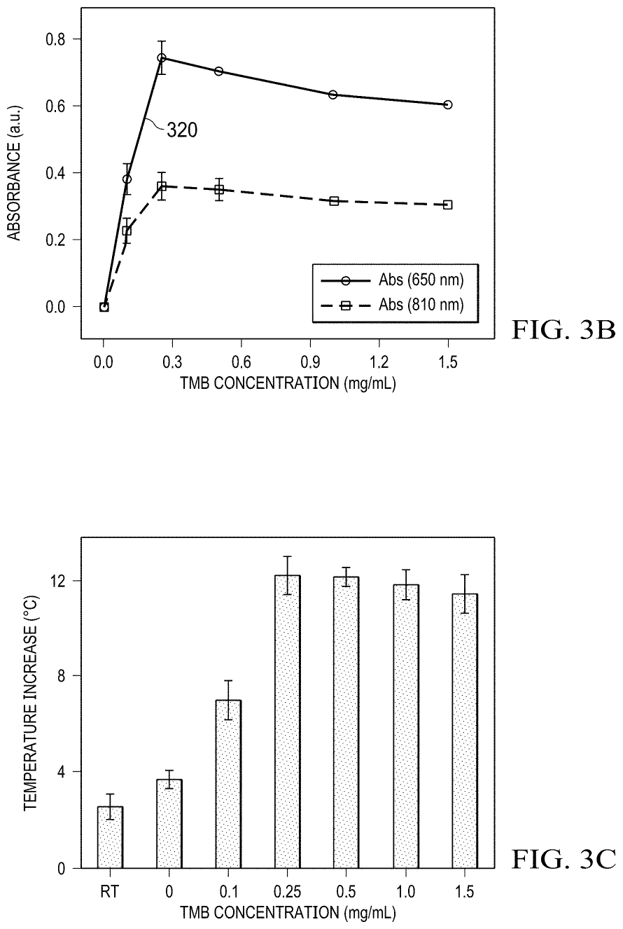

In this nanomaterial-mediated photothermal biosensing platform, TMB was used with the substrate to produce the photothermal biosensing probe (i.e., ox-TMB), and it is desirable for the concentration of TMB to be optimized in order to achieve the best detection performance. The off-chip UV-vis spectroscopy and on-chip temperature measurement were applied to characterize the optimization process. Generally, given a constant concentration of AuNPs (0.8 nM) and the reaction time (20 min), a series of TMB concentrations in the range from 0 to 1.5 mg/mL were tested. As seen in FIGS. 3A-B, the absorbances at 650 nm (representing the characteristic peak 310 of ox-TMB products) increased in the concentration range of 0-0.25 mg/mL and decreased at higher concentrations. The results indicated that, given a fixed amount of catalysts, the production of ox-TMB was enhanced when increasing substrate concentrations, and it reached the maximum amount when adding 0.25 mg/mL of TMB. When using excessive amounts of TMB, a slight color fading was found, which might be attributed to the formation of a light-yellow colored product because of its further oxidization. Similar changes were observed in the absorbances 320 at 810 nm in FIG. 3B (representing the typical absorption in the NIR region) with the maximum absorption obtained at 0.25 mg/mL, indicating potential NIR photothermal effects. Referring to FIG. 3C, under the laser irradiation, the temperature increased sharply from ΔT ˜2.0 to 12.0° C. at the TMB concentration from 0-0.25 mg/mL and reached a plateau (ΔT ˜12.0° C.) afterward, suggesting the maximum signals were obtained when the concentration of TMB was 0.25 mg/mL (FIG. 2C). Therefore, 0.25 mg/mL was used as the optimal TMB concentration in the following tests.

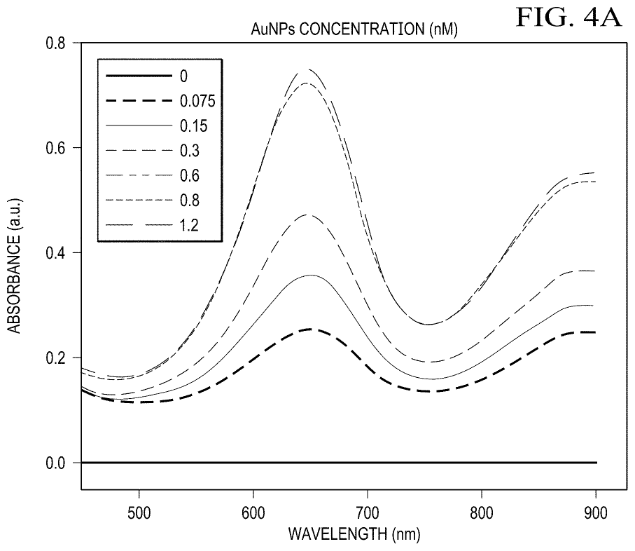

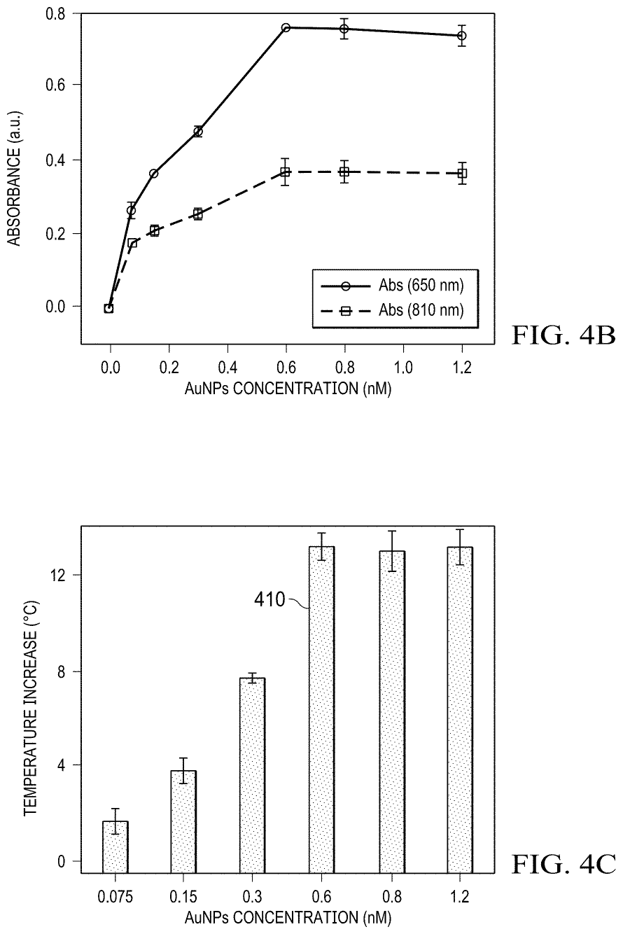

To obtain the maximum amount of ox-TMB, the concentration of the catalyst (AuNPs) in this TMB oxidization reaction system was also optimized for the best photothermal biosensing performance. By testing different concentrations (0-1.5 nM) of AuNPs, the off-chip UV-vis spectra and on-chip temperature measurement were applied to characterize the optimization process under the optimal concentration (0.25 mg/mL) of TMB. The absorbances at 650 nm and 810 nm were selected representing the typical peaks of the colorimetric and the NIR photothermal absorption. As shown in FIGS. 4A-4B, the absorbances at 650 nm increased from 0.075 to 0.6 nM, and no obvious change occurred when the concentration was higher than 0.6 nM. Therefore, it can be concluded that the saturated amount of ox-TMB was produced when adding 0.6 nM of AuNPs. Similarly, the absorbances at 810 nm increased in the range of 0.075-0.6 nM and reached a plateau afterward, indicating the maximum NIR absorption at the AuNPs concentration of 0.6 nM. Referring to FIG. 4C, upon laser irradiation, rapid temperature increases were observed when the concentration of AuNPs increased from 0.075 to 0.6 nM. The highest temperature increase with ΔT higher than 12.0° C. was achieved at the AuNPs concentration of 0.6 nM 410, and no significant changes in temperature elevations were recorded in the AuNPs concentration range of 0.6-1.2 nM. Consequently, the AuNPs concentration was optimized at 0.6 nM and used in the following experiments.

In the following AuNP-mediated photothermal biosensing of the target DNA, the DNA probe-AuNP conjugates instead of bare AuNPs were used as the catalyst for the photothermal biosensing probe (ox-TMB). It is worth noting that the DNA probe-AuNP conjugates were synthesized at a constant concentration ratio between the DNA probes and bare AuNPs. The characterization of the conjugates was conducted via UV-vis spectroscopy. A peak shift from 520 nm to 530 nm occurred for the conjugates in comparison with bare AuNPs (0.8 nM), which is attributed to the change of surface charges after bioconjugation with oligonucleotides.50 The concentration of AuNPs in the obtained conjugates was calculated using the Beer-Lambert law based on the extinction coefficient of 8.78×108 M-1·cm-1, and the concentration of conjugated DNA probes was confirmed using the NanoDrop spectrophotometer according to the absorbance at 260 nm. The final molar concentration ratio of the DNA probe-AuNP conjugates was obtained as 220:1 (DNA probes: AuNPs) in the photothermal genetic analysis platform.

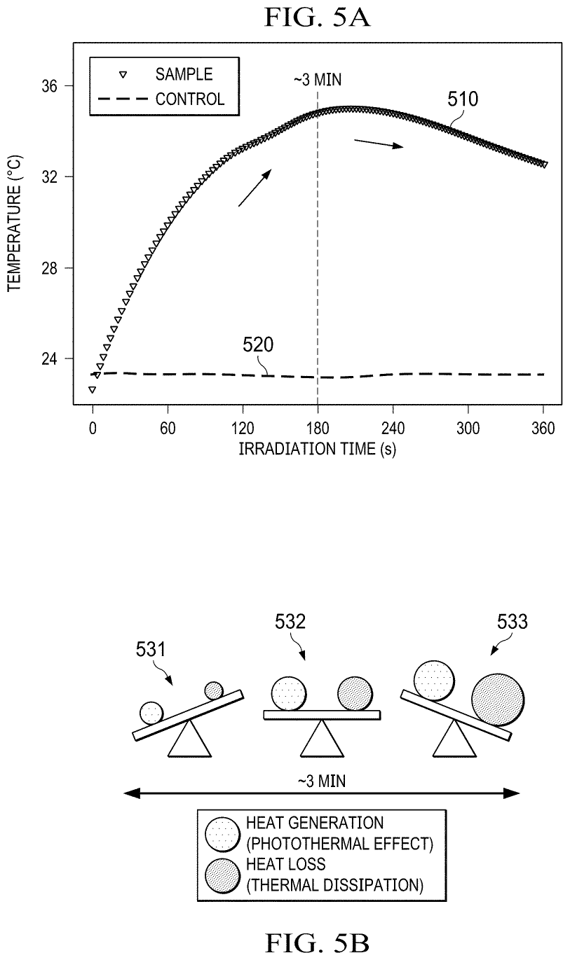

To characterize the effect of the irradiation time and obtain the maximum temperature signals, kinetic studies were conducted for the photothermal biosensing of the target DNA. Under continuous laser irradiation, the dynamic temperature changes of both the control 510 (in the absence of target DNA) and a sample 520 (in the presence of 10 μM target DNA) were monitored for 6 min. The results are shown in FIG. 5A, and the effects of different factors on the photothermal measurement are illustrated in FIG. 5B. In this embodiment, before 3 minutes 531 heat generation is greater than heat loss, at approximately 3 minutes 532 heat generation is approximately equal to heat loss, and beyond 3 minutes 533 heat generation is less than heat loss. There was no obvious temperature increase found in the control as compared to room temperature (˜23.0° C.). In the presence of the target DNA, a rapid temperature increase was observed from 23.0 to 33.0° C. in the first 120 s due to the strong photothermal conversion. From 120 s to 180 s, the temperature of the sample increased slowly, which might be due to enhanced heat loss resulted from a greater temperature gradient between the sample and the surroundings, as a higher sample temperature was achieved than before. At around 3 min, the temperature reached the highest value of ˜35.0° C., suggesting the balance between heat generation (due to the photothermal effect of ox-TMB) and heat loss (due to thermal dissipation). After 3 min, the temperature began gradually decreasing, possibly because photothermal conversion became saturated and heat loss became the predominant factor. Therefore, to achieve the sensitive photothermal biosensing of target DNA, the laser irradiation time of 3 min was used in the following experiments.

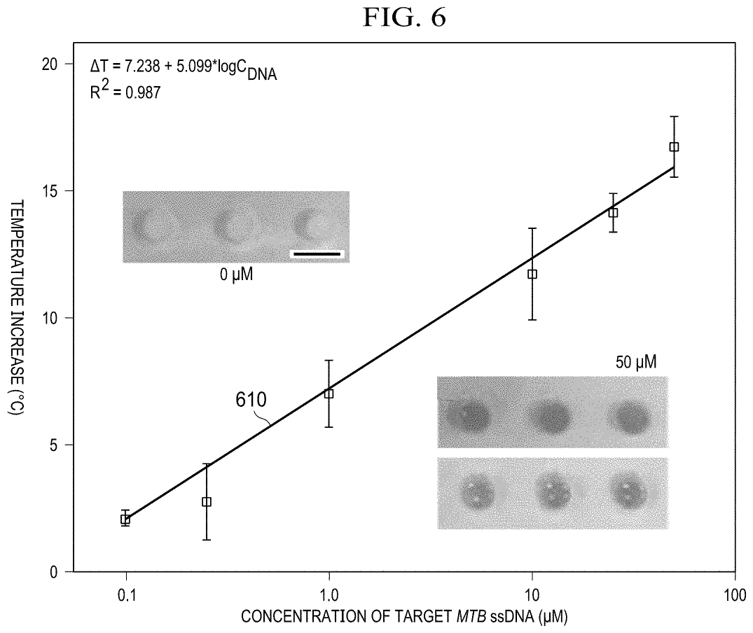

Under optimal conditions, the on-chip photothermal detection of MTB DNA was performed by recording temperature increases of samples using a thermometer. A series of different concentrations in the range of 0-50 μM for synthetic MTB DNA samples were tested. Insets (a-b) in FIG. 6 show that blue color, inset (b), was clearly observed when testing the target DNA (such as at the concentration of 50 μM), while no color change, inset (a), was observed in the absence of the target DNA (0 μM). In the photothermal biosensing results, the temperature of samples increased when adding higher concentrations of the target and reached a ΔT value of nearly 17.0° C. at 50 μM of target DNA. The plot 610 in FIG. 6 shows a linear relationship between temperature increases and the logarithmic concentrations of the synthetic target DNA in the range of 100 nM to 50 μM. The square of the correlation coefficient was 0.987, with a slope of 5.099° C.·μM⁻1. The limit of detection (LOD) was calculated to be 39 nM (or 0.58 μg/mL) based on the 3-fold standard deviation over the blank.

It is noted that in comparison with conventional colorimetric biosensing methods, there are several significant advantages in our photothermal detection method. First, the proposed method provides higher sensitivity for the detection of target DNA, obtaining a lower LOD value (0.58 μg/mL) than those reported based on colorimetric signals (LODs: 10 μg/mL, 1.88 μg/mL, or 1.14 μg/mL). In addition, with only a simple and inexpensive signal reader (a thermometer), quantitative detection of DNA can be achieved, avoiding the need for bulky and expensive instruments (such as spectrometers) and significantly reducing the bioassay cost. Furthermore, the quantification of DNA is based on temperature readouts, thereby avoiding color interference from the sample matrix, which is usually a common problem in colorimetric biosensing methods in testing colored real samples, such as blood matrices. Herein, we used a food dye (red color) to mimic the real colored matrix of a blood sample, and observation of expected blue colored ox-TMB products was interfered with remarkably due to the red colored mimic matrix background. For instance, the inset (c) in FIG. 6 shows a pink color instead of the blue color typically shown when testing initially colorless samples. We did not see such an interference problem in our thermometer-based method, which is another advantage of our method over the colorimetric method.

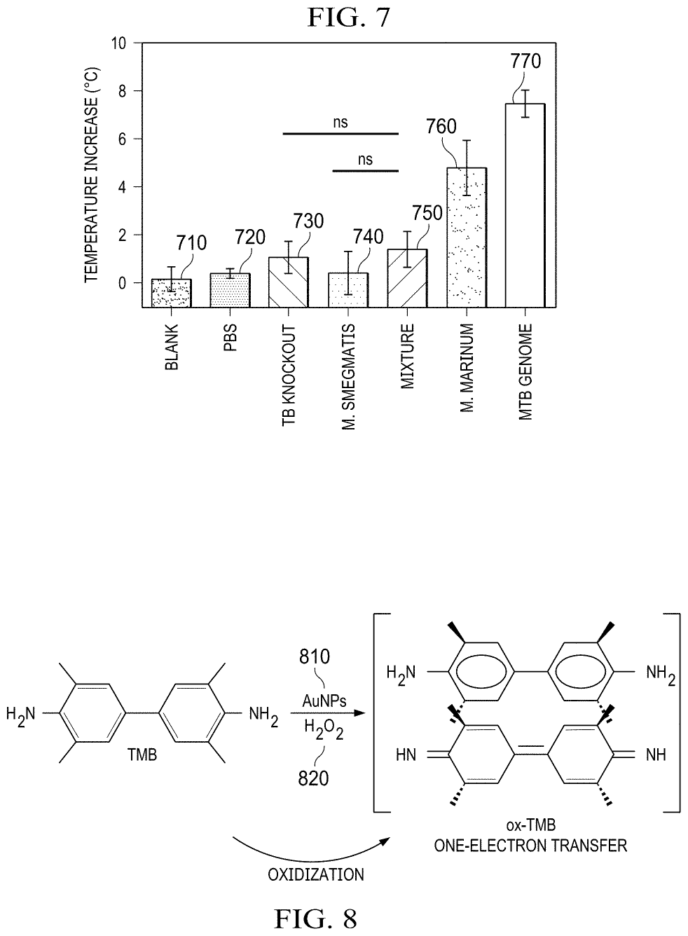

Referring to FIG. 7, the on-chip photothermal biosensing method was further validated by investigating the specificity for the detection of genomic DNA instead of synthetic sequences. In addition to MTB genomic DNA 770, other interfering species were used, including water as blank 710, PBS buffer 720, TB knockout DNA (with the deletion of EsxB:EsxA from MTB) 730, M. smegmatis (a non-pathogenic mycobacterium that has been widely used as an alternative for MTB due to the fast growth and the requirement of low biosafety level facility) 740, a DNA mixture from the above species (M. smegmatis and TB knockout) 750, and M. marinum (a pathogenic non-tuberculous mycobacterium) 760. As shown in FIG. 7, a significant temperature increase of approximately 8.0° C. was acquired in the detection of MTB genomic DNA 770 with the analytical recovery of 113±1%, even at a 2-fold lower concentration than others, while neglectable temperature increases were obtained from blank, PBS buffer, TB knockout DNA, and M. smegmatis DNA. Even when testing a mixture of DNA interference samples containing M. Smegmatis and TB knockout, the photothermal biosensing signals remained similar to those from individual components, indicating high specificity of our method. It was noted that the sample containing M. marinum genomic DNA at 2-fold higher concentrations had a mild temperature increase of 5.0° C., which was mainly due to a high percent identity (over 80%) in genomes between MTB and M. marinum. Therefore, it can be concluded that the proposed photothermal biosensing method has high specificity even when distinguishing interfering substances with high similarity.

Referring to FIG. 8, the oxidation reaction of TMB to ox-TMB in the presence of AuNPs 810 and H₂O₂ 820 is illustrated. In this embodiment, the basis of quantitative photothermal detection is that the oxidized detector probe evolves heat when exposed to NIR actinic energy.

Referring to FIG. 9, absorption spectra for AuNPs and conjugated DNA-AuNPs are illustrated. The AuNPs spectra 910 shows an absorption peak at approximately 520 nm. The conjugated DNA-AuNPs spectra 920 shows an absorption peak at approximately 530 nm.

Illustrative embodiments provide a low-cost photothermal biosensing method for visual quantitative nucleic acid detection on a paper hybrid device using a thermometer. By applying the AuNP-mediated photothermal effect in bioassays, the target DNA is quantitatively detected using temperature signals as analytical readouts, achieving higher sensitivity with no color interference, contrasting that from conventional colorimetric detection methods. The entire assay for the quantitative detection of MTB DNA as a model target can be completed within 2 h on a low-cost paper/polymer hybrid device (the material cost of $0.08 for each device), without the need for any costly instrumentation and complicated nucleic acid amplification procedures. This method was further validated by detecting genomic DNA with high specificity. Illustrative embodiments perform photothermal genetic analysis on paper hybrid microfluidic devices, providing a simple, low-cost, rapid, and quantitative photothermal microfluidic biosensing platform. With the rapid development of commercially available portable lasers, the portability of this photothermal platform will be further enhanced.

Since this photothermal genetic biosensing platform is based on nucleic acid hybridization, it may be useful in a wide range of biological applications based on conventional DNA hybridization techniques such as DNA microarray. Although the DNA microarray technique can provide high throughput, it usually requires costly fluorescence scanners. The illustrative embodiments outperforms conventional DNA microarray (e.g. using glass slides as substrates) in genetic analysis in terms of the aspects of simplicity, ease of operation, affordability, etc. The combination of all these significant features with a low-cost and portable paper hybrid microfluidic device make it particularly suitable for POC applications. Many new complementary genetic assays using a thermometer as the signal reader may be developed. Overall, considering genetic analysis is widely used in various biological applications including infectious disease diagnosis, this photothermal biosensing platform has great potential for broad applications, such as POC disease diagnosis, especially in resource-poor settings.

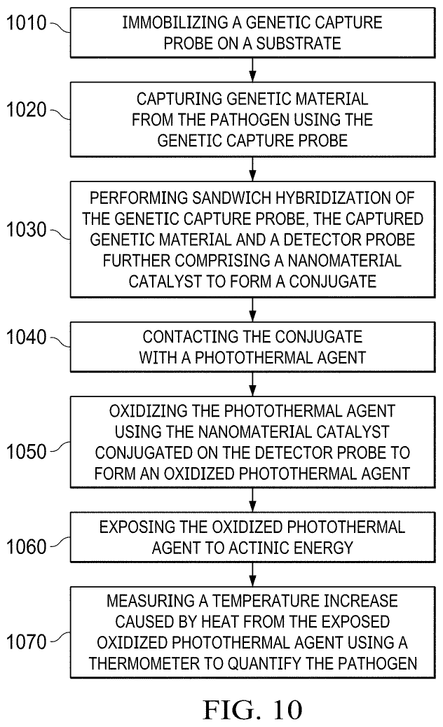

FIG. 10 shows a flow chart of a process that can be implemented by a computer program. The process can include quantitative genetic detection of a pathogen. The process can begin with immobilizing a genetic capture probe on a substrate 1010. The process can then include capturing genetic material from the pathogen using the genetic capture probe 1020. The process can then include performing sandwich hybridization of the genetic capture probe, the captured genetic material and a detector probe further comprising a nanomaterial catalyst to form a conjugate 1030. The process can then include contacting the conjugate with a photothermal agent 1040. The process can then include oxidizing the photothermal agent using the nanomaterial catalyst conjugated on the detector probe to form an oxidized photothermal agent 1050. The process can then include exposing the oxidized photothermal agent to actinic energy 1060. The process can then include measuring a temperature increase caused by heat from the exposed oxidized photothermal agent using a thermometer to quantify the pathogen 1070.

The description of the different illustrative embodiments has been presented for purposes of illustration and description and is not intended to be exhaustive or limited to the embodiments in the form disclosed. The different illustrative examples describe components that perform actions or operations. In an illustrative embodiment, a component can be configured to perform the action or operation described. For example, the component can have a configuration or design for a structure that provides the component an ability to perform the action or operation that is described in the illustrative examples as being performed by the component. Further, To the extent that terms “includes”, “including”, “has”, “contains”, and variants thereof are used herein, such terms are intended to be inclusive in a manner similar to the term “comprises” as an open transition word without precluding any additional or other elements.

The descriptions of the various embodiments of the present invention have been presented for purposes of illustration, but are not intended to be exhaustive or limited to the embodiments disclosed. Not all embodiments will include all of the features described in the illustrative examples. Further, different illustrative embodiments may provide different features as compared to other illustrative embodiments. Many modifications and variations will be apparent to those of ordinary skill in the art without departing from the scope and spirit of the described embodiment. The terminology used herein was chosen to best explain the principles of the embodiment, the practical application or technical improvement over technologies found in the marketplace, or to enable others of ordinary skill in the art to understand the embodiments disclosed here.

What is claimed is:

1. A method of quantitative genetic detection of a pathogen, comprising:
immobilizing a genetic capture probe on a substrate;
capturing genetic material from the pathogen using the genetic capture probe;
performing sandwich hybridization of the genetic capture probe, the captured genetic material and a detector probe further comprising a nanomaterial catalyst to form a conjugate;
contacting the conjugate with a photothermal agent;
oxidizing the photothermal agent using the nanomaterial catalyst conjugated on the detector probe to form an oxidized photothermal agent;
exposing the oxidized photothermal agent to actinic energy; and
measuring a temperature increase caused by heat from the exposed oxidized photothermal agent using a thermometer to quantify the pathogen.

2. The method of claim 1, wherein the pathogen further comprises Mycobacterium tuberculosis.

3. The method of claim 1, wherein the genetic capture probe further comprises a DNA capture probe.

4. The method of claim 1, wherein the substrate further comprises paper located within a paper hybrid microfluidic device.

5. The method of claim 4, wherein the genetic capture probe is immobilized on the paper through a one-step surface modification process.

6. The method of claim 1, wherein the genetic material from the pathogen further comprises target DNA.

7. The method of claim 1, wherein the nanomaterial catalyst further comprises at least one member selected from the group consisting of oligonucleotide-functionalized gold nanoparticles (AuNPs), oligonucleotide-functionalized iron nanoparticles (Fe₃O₄NPs), oligonucleotide-functionalized platinum nanoparticles (PtNPs).

8. The method of claim 1, wherein the photothermal agent further comprises 3,3′,5,5′-tetramethylbenzidine (TMB).

9. The method of claim 1, wherein exposing the oxidized photothermal agent to actinic energy further comprises exposing the oxidized photothermal agent to near infrared laser irradiation.

10. An apparatus for quantitative genetic detection of a pathogen, comprising:
a substrate;
a genetic capture probe immobilized on the substrate;
genetic material from the pathogen captured by the genetic capture probe;
a detector probe sandwich hybridized with the captured genetic material from the pathogen and the capture probe, the detector probe further comprising a nanomaterial catalyst to form a conjugate;
a photothermal agent oxidized using the nanomaterial catalyst conjugated on the detector probe;
a laser configured to expose the oxidized photothermal agent to actinic energy; and
a thermometer configured to measure a temperature increased cause by heat from the exposed oxidized photothermal agent to quantify the pathogen.

11. The apparatus of claim 10, wherein the pathogen comprises Mycobacterium tuberculosis.

12. The apparatus of claim 10, wherein the genetic capture probe further comprises a DNA capture probe.

13. The apparatus of claim 10, wherein the substrate further comprises paper located within a paper hybrid microfluidic device.

14. The apparatus of claim 10, wherein the genetic material from the pathogen further comprises target DNA.

15. The apparatus of claim 10, wherein the nanomaterial catalyst further comprises at least one member selected from the group consisting of oligonucleotide-functionalized gold nanoparticles (AuNPs), oligonucleotide-functionalized iron nanoparticles (Fe₃O₄NPs), oligonucleotide-functionalized platinum nanoparticles (PtNPs).

16. The apparatus of claim 10, wherein the photothermal agent further comprises 3,3′,5,5′-tetramethylbenzidine (TMB).

17. The apparatus of claim 10, wherein the laser is configured to generate near infrared laser irradiation.

18. A device for quantitative genetic detection of a pathogen, comprising:
a substrate;
a genetic capture probe immobilized by the substrate;
genetic material from the pathogen captured on the genetic capture probe;
a detector probe sandwich hybridized with the captured genetic material from the pathogen and the capture probe, the detector probe further comprising a nanomaterial catalyst to form a conjugate; and
a photothermal agent oxidized using the nanomaterial catalyst conjugated on the detector probe.

19. The device of claim 18, wherein the substrate further comprises paper located within a paper hybrid microfluidic device.

20. The device of claim 18,
wherein the genetic material from the pathogen further comprises target DNA,
wherein the nanomaterial catalyst further comprises at least one member selected from the group consisting of oligonucleotide-functionalized gold nanoparticles (AuNPs), oligonucleotide-functionalized iron nanoparticles (Fe₃O₄NPs), oligonucleotide-functionalized platinum nanoparticles (PtNPs) and
wherein the photothermal agent further comprises 3,3′,5,5′-tetramethylbenzidine (TMB).
